(19) United States
(12) Patent Application Publication
(10) Pub. No.: US 2006/0223788 A1
(43) Pub. Date: Oct. 5, 2006

(54) Analgesic composition for topical use

(76) Inventor: Clifton H. Cathcart, Lufkin, TX (US)

Correspondence Address: CONLEY ROSE, P.C., P. O. BOX 3267, HOUSTON, TX, 77253-3267, US

(21) Appl. No.: 11/092,394

(22) Filed: Mar. 29, 2005

(51) Int. Cl. A61K 31/573; A61K 31/198; A61K 31/542; A61K 31/24

(52) U.S. Cl. 514/171; 514/561; 514/537

(57) ABSTRACT

An analgesic composition, is disclosed which comprises a mixture of piroxicam, dexamethasone, ketamine, lidocaine injection, dimethyl sulfoxide, gabapentin and Vanicream™, preferably in the form of a cream or ointment. The composition is applied topically for the relief of pain of arthritis, neuropathy, post-herpetic (shingles) conditions, sore muscles, tendons and ligaments, and local reactions to insect bites or stings.

### CROSS-REFERENCE TO RELATED APPLICATIONS

Not Applicable.

### STATEMENT REGARDING FEDERALLY SPONSORED RESEARCH OR DEVELOPMENT

Not Applicable.

### BACKGROUND OF THE INVENTION

1. Technical Field of the Invention

The present invention generally relates to compositions and methods for alleviating pain in mammals. More particularly, the invention relates to such methods which include topical application of the composition to the skin of the mammal.

2. Description of Related Art

Local pain can result from any of a variety of causes such as body injury, infection or disease, inflammation, muscle spasm and neuropathy. Examples of conditions that are typically associated with localized pain in the skin or in a tissue or structure near the skin include arthritis, neuropathy, post-herpetic (shingles) conditions, a sore muscle, tendon or ligament, and a local reaction to an insect bite or sting. Typically, a sensation of pain occurs when free nerve endings that constitute pain receptors in the skin or internal tissue are subjected to a mechanical, thermal or chemical stimulus. The stimulus causes the pain receptors to transmit a responsive signal along afferent nerves to the central nervous system and then on to the brain. When pain persists or recurs frequently and treatment provides insufficient relief, in addition to the primary discomfort, the person may be further debilitated by limited function (e.g., of an arthritic joint), reduced mobility, interrupted sleep, and a generally diminished quality of life.

Many topically applied analgesic drugs have been described to treat the symptoms of pain, most of which have very limited or short-lived analgesic effects. For mild relief of minor discomfort, there are a variety of non-prescription topical preparations in common use today which contain counter-irritants such as camphor, menthol, capsaicin, and eucalyptus. As noted in U.S. Pat. No. 6,723,345 (Drizen et al.), such products are not intended for deep penetration of tissue structures. Pain that is more problematic, however, requires a more effective analgesic agent. Among the therapeutic preparations that are available by prescription are EMLA™ cream, which contains 2.5% lidocaine-prilocaine, and LIDODERM™ patch. Some other analgesics that have been tried include a combination of DMSO and ibuprofen, and a combination of Speedgel™, ibuprofen, ketamine, lidocaine and dexamethasone.

U.S. Pat. No. 6,461,600 (Ford) notes that the usefulness of topical pain relief medicaments is highly dependent on the selection of a suitable carrier. A certain cream carrier for topical delivery of medicaments including certain analgesics is described.

U.S. Pat. No. 6,455,066 (Fischer et al.) describes the usefulness of penetration agents to assist intradermal and transdermal drug administration due to the skin's intrinsic resistance to drug penetration. It is said that topical application of local anesthetics has not been widely used primarily because of the difficulty of getting significant concentrations of local anesthetics through the skin barrier without also causing increased systemic absorption when a penetration agent is employed. A triglyceride and an aloe composition are described as intradermal-penetration agents. The duration of a local anesthetic's effect is said to be augmented by the penetration properties of the triglyceride or aloe composition.

U.S. Pat. No. 6,638,981 (Williams et al.) describes certain topical compositions and methods for treating pain using certain oil-in-water emulsions comprising an antidepressant, an N-methyl-D-aspartate receptor antagonist such as ketamine, a lipophilic component, water and a surfactant.

U.S. Pat. No. 5,817,699 (Flores et al.) describes a certain process for producing a ketamine ointment. It is said that the ointment can be directly applied to a subject's pain site thereby rapidly alleviating the pain while avoiding ketamine's side-effects. The primary ingredients in the ointment are said to be ketamine hydrochloride, lecithin organogel, ethoxy diglycol (Carbitol™), pluronic F-127 gel, and deionized distilled water.

U.S. Pat. No. 6,537,991 (Shaw et al.) describes certain methods and compositions employing an antagonist of the pain-enhancing effects of E-type prostaglandins for prevention and treatment of neuropathic pain. Combination therapy may involve the use of a locally applied local anesthetic.

Despite the advancements that have been made in the treatment of pain, there remains a need for a therapeutic agent that can be conveniently topically applied to the skin to provide effective and longer lasting pain relief or pain reduction.

### SUMMARY OF THE INVENTION

In accordance with certain embodiments of the present invention, an analgesic composition for alleviating pain is provided which comprises a pain alleviating mixture resulting from the combination of piroxicam, dexamethasone, ketamine, lidocaine, DMSO, gabapentin and Vanicream™. In certain embodiments the mixture comprises 0.5 wt % piroxicam; 0.4 wt % dexamethasone; 1 wt % ketamine; 0.5 vol/wt % lidocaine injection (2% solution); 10 vol/wt % dimethylsulfoxide; 1-2 wt % gabapentin; and 85.6-86.6 wt % Vanicream™. Preferably the composition is in the form of a cream or ointment.

Also provided in accordance with the present invention is a method of making the above-described pain-relieving composition comprising mixing together (a) 4.8 grams piroxicam powder, finely ground; (b) 1.92 grams dexamethasone powder; (c) 4.8 grams ketamine powder; (d) 120 milliliters of a 2% solution of lidocaine injection; (e) 48 milliliters dimethylsulfoxide; (f) 4.5-9 grams gabapentin powder; and (g) 16 ounces (453.6 grams) Vanicream™.

In accordance with another embodiment of the present invention, a method of treating pain is provided that includes topically administering to the skin at a site of pain in an individual in need thereof, a pain alleviating amount of an above-described pain relieving composition or mixture. In some embodiments the individual is suffering from at least one of the following: arthritic pain, neuropathic pain; muscle, tendon or ligament pain; post herpetic pain; or a painful local reaction to an insect bite or sting.

In some embodiments, the method comprises rubbing about 0.5-1.0 gram of the above-described composition onto the skin of the individual at the painful site on the body. In some embodiments, the method includes applying a pain relieving amount of the composition 2-3 times daily. These and other embodiments, features and advantages of the present invention will be apparent from the following detailed description.

### DETAILED DESCRIPTION OF THE PREFERRED EMBODIMENTS

Definitions:

As used herein, the term “analgesic” refers to any member of the diverse group of drugs used to relieve pain. This includes, but is not limited to, drugs that primarily reduce the perception of pain as well as drugs which primarily block the sensation of pain by numbing the nerves that transmit pain to the brain.

Alleviation of pain refers to partially or completely stopping pain, which includes but is not limited to blocking, relieving or lessening a pain sensation.

The term “pain-alleviating” shall be understood herein to include the expressions “pain-suppressing” and “pain-inhibiting” as the invention is applicable to the alleviation of existing pain as well as the suppression or inhibition of pain which would otherwise ensue from an imminent pain-causing event.

A new composition or ointment that is useful for topical application to relieve or ameliorate pain includes the following ingredients: piroxicam, dexamethasone, ketamine; lidocaine; dimethyl sulfoxide liquid (DMSO); gabapentin; and Vanicream™. All of the pharmaceutical components can be readily obtained from any major pharmaceutical supplier by a licensed physician or pharmacist. Vanicream™ is a commercially available skin moisturizing cream which is an oil-in-water emulsion type vanishing cream base composed of purified water, white petrolatum, cetearyl alcohol and ceteareth-20, sorbitol solution, propylene glycol, simethicone, glyceryl monostearate, polyethylene glycol monostearate, sorbic acid and BHT. Although the commercial product known as Vanicream™ is preferred, another preparation of similar composition or properties could be substituted if desired.

### EXAMPLE 1

### Preparation of N-6 Neuro Pain Cream

A preferred neuro pain cream (also denoted herein as N-6) is prepared by placing 4.8 grams Piroxicam into a bowl and grinding the piroxicam to a fine powder. To that fine powder is added 1.92 grams dexamethasone powder, 4.8 grams Ketamine powder, 120 milliliters lidocaine 2% injection (115.01 g), 48 milliliters of a 10% solution of dimethyl sulfoxide (45.9 g), 4.5 grams gabapentin powder, and 16 ounces (453.6 grams) Vanicream™. The ingredients are thoroughly mixed, to yield approximately 638 grams of therapeutic cream comprising having the following approximate composition:

| | | |
|---|---|---|
| 0.5 | wt % | piroxicam |
| 0.4 | wt % | dexamethasone |
| 1 | wt % | ketamine |
| 0.5 | vol % | lidocaine |
| 10 | vol % | DMSO |
| 1 | wt % | gabapentin |
| 86.6 | wt % | Vanicream ™ |

The above-stated percentages are based on the weight of the final composition. If desired, the amount of gabapentin may be increased to 2 wt %. The amounts of these components may be proportionally scaled up or down to produce the desired total quantity of pain cream.

### EXAMPLE 2

### Therapeutic Use of the Neuro Pain Cream

The composition prepared as described in Example 1 was self-administered by a test group of patients, topically applying about 0.5 to 1 gram to a painful area, rubbing the cream over the painful area and into the skin, preferably 2-3 times daily, as needed. Preferably the dosage used is sufficient to relieve or reduce pain, but less than an amount at which an undesirable side effect from a component of the composition can occur. Use of this composition on children is generally not recommended.

Out of a total test group of 272 adult patients who tried the new neuro pain cream, survey results were obtained from 99 patients. The painful conditions of those patients varied in degree of pain, and comprised one or more of the following conditions: arthritic, neuropathic, muscle, tendon, and ligament pain, as well as post herpetic (shingles) pain and local reactions to insect bites or stings. The subjective results were reported by the patients as follows:

| | Number of | Percentage of |
|---|---|---|
| Level of Pain Relief | Responses | Total Responses |
| No relief | 6 | 6% |
| Slight relief | 24 | 24% |
| Good relief | 45 | 46% |
| Excellent relief | 23 | 23% |
| Unsure | 1 | 1% |

In summary, 70% of the individuals who responded reported good to excellent results. Each reported level of pain relief was a subjective evaluation by the patient, e.g., with each patient comparing the new pain treatment cream to various products they may have used similarly in the past. The average length of time that pain relief lasted was 6-8 hours. No side effects were reported.

### EXAMPLE 3

### Representative Case Studies

In one case, the patient applied the above-described neuropain cream to a painful area twice daily for approximately once a week and reported slight relief that lasted about 1 hour. The pain cream also reportedly worked well in combination with use of a Homedics massager.

In another representative case, the patient applied the neuropain cream daily to a painful area, one application per day. Excellent pain relief was reported, lasting more than 12 hours. The cream was considered advantageous because it was non-greasy, odor free and did not rub off on the patient's clothes.

Another representative patient reported using the cream five days a week, applied one to two times a day, and obtained good pain relief which lasted for 6-8 hours. The advantages of direct application to the painful site and lack of side effects were noted, in contrast to other pain medications that are taken by mouth.

Still another representative patient reported using the neuropain cream every day, applying the cream two or more times a day. Good pain relief and excellent pain relief were reported, with relief lasting about 2-6 hours, depending on the severity of the pain. The new pain cream reportedly worked better than other pain rubs the patient had used.

The above-described analgesic composition and method of treatment are applicable for alleviating pain in a variety of conditions, including, but not limited to, arthritic joint pain, neuropathic pain, post-herpetic (shingles) conditions, a sore muscle, tendon or ligament, and local reactions to an insect bite or sting. Without wishing to be limited to any particular theory to explain the beneficial results that are obtained, it is believed that the above-described pain cream penetrates the skin and reduces the painful inflammatory response in underlying tissues and structures. It is also proposed that this composition also blocks pain receptors, and reduces inflammation and muscle spasms. The present composition provides more effective and longer lasting pain relief or pain reduction than most other similarly applied pain relief products. It also provides a greater scope of therapeutic effect than many conventional products. The pain cream is also essentially odorless, and it does not impart an abnormal sensation on the skin, (e.g., numbness or tingling). The new pain cream also has advantages over conventional pain relief therapies that require ingestion or injection of a medication, because it is easier to use (i.e., direct application to the skin) and because of minimal or no systemic side effects.

What is claimed is:

1. An analgesic mixture comprising:
Piroxicam;
Dexamethasone;
Ketamine;
Lidocaine injection;
Dimethyl sulfoxide;
Gabapentin; and
Vanicream™.

2. The analgesic mixture of claim 1 comprising:
0.5 wt % piroxicam;
0.4 wt % dexamethasone;
1 wt % ketamine;
0.5 vol/wt % lidocaine injection (2% solution);
10 vol/wt % dimethylsulfoxide;
1-2 wt % gabapentin; and
85.6-86.6 wt % Vanicream™.

3. The analgesic mixture of claim 2 comprising 1 wt % gabapentin.

4. The analgesic mixture of claim 1 prepared by a process that comprises mixing together:
(a) 4.8 grams piroxicam powder, finely ground;
(b) 1.92 grams dexamethasone powder;
(c) 4.8 grams ketamine powder;
(d) 120 milliliters of a 2% solution of lidocaine injection;
(e) 48 milliliters dimethylsulfoxide;
(f) 4.5-9 grams gabapentin powder; and
(g) 16 ounces (453.6 grams) Vanicream™.

5. A method of treating pain in an individual in need of such treatment comprising topically administering to the skin of said individual at a site of pain, a pain alleviating amount of the mixture of claim 1.

6. The method of claim 5 wherein said individual is suffering from arthritic pain.

7. The method of claim 5 wherein said individual is suffering from neuropathic pain.

8. The method of claim 5 wherein said individual is suffering from muscle, tendon or ligament pain.

9. The method of claim 5 wherein said individual is suffering from post herpetic pain.

10. The method of claim 5 wherein said individual is suffering from a painful local reaction to an insect bite or sting.

11. The method of claim 5 comprising rubbing about 0.5-1.0 gram of the mixture of claim 1 onto the skin at said site.

12. The method of claim 5 comprising applying a pain relieving amount of said mixture to the skin at said site 2-3 times daily.
